(19) United States
(12) Patent Application Publication
(10) Pub. No.: US 2018/0110474 A1
(43) Pub. Date: Apr. 26, 2018

(54) MEASUREMENT DEVICE AND MEASUREMENT METHOD

(71) Applicant: SEIKO EPSON CORPORATION, Tokyo (JP)

(72) Inventors: Akiko YAMADA, Shiojiri-shi (JP); Kohei YAMADA, Shiojiri-shi (JP)

(73) Assignee: SEIKO EPSON CORPORATION, Tokyo (JP)

(21) Appl. No.: 15/718,713

(22) Filed: Sep. 28, 2017

(30) Foreign Application Priority Data
Oct. 24, 2016 (JP) 2016-207615

(51) Int. Cl. A61B 5/00; A61B 5/02; A61B 5/026

(52) U.S. Cl. CPC A61B 5/7278; A61B 5/02007; A61B 5/0004; A61B 5/0261; A61B 5/742; A61B 5/681

(57) ABSTRACT

A measurement device includes: a calculation unit that calculates a blood flow index regarding a waveform of a blood flow signal indicating a temporal change in a blood flow rate of a subject; and a determination unit that determines whether there is stenosis or atresia of a blood vessel of the subject according to the blood flow index.

### BACKGROUND

### 1. Technical Field

The present invention relates to a technology for evaluating an intravascular state of a subject.

### 2. Related Art

Various technologies for evaluation body states of subjects have been proposed in the related art. For example, JP-A-11-104090 discloses a configuration for evaluating a cardiac function state of a subject from a pulse wave pattern of the subject.

To evaluate body states, characteristics of arteries can be used. Incidentally, for example, stenosis or atresia of a blood vessel due to occurrence of blood clots or atheroma in the blood vessel may cause cerebral infarction or cardiac infarction. Accordingly, from the viewpoint of predicting diseases such as cerebral infarction or cardiac infarction, it is particularly important to determine whether there is stenosis or atresia of a blood vessel due to occurrence of blood clots or atheroma in the blood vessel. However, JP-A-11-104090 does not mention evaluation of whether there is stenosis or atresia of a blood vessel due to occurrence of blood clots or atheroma in the blood vessel.

### SUMMARY

An advantage of some aspects of the invention is to determine that there is stenosis or atresia of a blood vessel of a subject.

A measurement device according to a preferred aspect of the invention includes: a calculation unit that calculates a blood flow index regarding a waveform of a blood flow signal indicating a temporal change in a blood flow rate of a subject; and a determination unit that determines whether there is stenosis or atresia of a blood vessel of the subject according to the blood flow index. According to the above configuration, it is possible to determine whether there is the stenosis or atresia of the blood vessel of the subject according to the blood flow index.

In the preferred aspect of the invention, the calculation unit may calculate the blood flow index according to a hemline shape of a peak of the waveform. In the above configuration, the blood flow index is calculated according to the hemline shape of the peak of the waveform of the blood flow signal. The hemline shape of the peak of the waveform of the blood flow signal has a tendency to differ between a case in which stenosis or atresia of a blood vessel occurs and a normal case (that is, a case in which no stenosis or atresia of a blood vessel occurs). According to the above-described aspect of the invention, it is possible to determine whether there is the stenosis or atresia of the blood vessel based on the above tendency.

In the preferred aspect of the invention, the calculation unit may calculate the blood flow index according to kurtosis of the waveform. In the above configuration, the blood flow index according to the kurtosis of the waveform of the blood flow signal is calculated. The kurtosis of the waveform of the blood flow signal at the stenosis time or the atresia time of the blood vessel has a tendency to be less than at the normal time. According to the above-described aspect of the invention, whether there is the stenosis or atresia of the blood vessel can be determined with high precision based on the above tendency.

In the preferred aspect of the invention, the calculation unit may calculate the blood flow index according to a half width of the waveform. In the above configuration, the blood flow index according to the half width of the waveform of the blood flow signal is calculated. The half width of the waveform of the blood flow signal at the stenosis time or the atresia time of the blood vessel has a tendency to be greater than at the normal time. According to the above-described aspect of the invention, whether there is the stenosis or atresia of the blood vessel can be determined with high precision based on the above tendency.

In the preferred aspect of the invention, the calculation unit may calculate the blood flow index according to a waveform in a unit section equivalent to one beat of pulsation in the blood flow signal. In the above configuration, the blood flow index according to the waveform in the unit section equivalent to one beat of pulsation in the blood flow signal is calculated. A change in the waveform according to whether there is the stenosis or atresia of the blood vessel tends to be more remarkable at one beat of pulsation. Thus, according to the above-described aspect of the invention, whether there is the stenosis or atresia of the blood vessel can be determined with high precision based on the above tendency.

In the preferred aspect of the invention, the calculation unit may calculate the blood flow index according to a ratio between a first peak strength and a second peak strength in a unit section equivalent to one beat of pulsation in the blood flow signal. In the above configuration, the blood flow index is calculated according to the ratio between the first peak strength and the second peak strength in the unit section equivalent to one beat of the pulsation in the blood flow signal. The second peak strength with respect to the first peak strength in the unit section at the stenosis time or the atresia time of the blood vessel has a tendency to be less than at the normal time. According to the above-described aspect of the invention, whether there is the stenosis or atresia of the blood vessel can be determined with high precision based on the above tendency.

In the preferred aspect of the invention, the calculation unit may calculate the blood flow index from an average waveform obtained by averaging waveforms of a plurality of unit sections in which the blood flow signal is divided at each pulsation over a period of the plurality of unit sections. In the above configuration, the blood flow index is calculated from the average waveform obtained by averaging the waveforms of the plurality of unit sections in which the blood flow signal is divided at each pulsation over a period of the plurality of unit sections. Accordingly, the blood flow index can be calculated in accordance with a smoothed waveform compared to a configuration in which the blood flow index is calculated from the waveform in one unit section. Further, whether there is the stenosis or atresia of the blood vessel can be determined with higher precision.

In the preferred aspect of the invention, the calculation unit may calculate the blood flow index by calculating an index regarding a waveform of the unit section in each of the plurality of unit sections in which the blood flow signal is divided at each pulsation and averaging the indexes over a period of the plurality of unit sections. In the above configuration, the blood flow index is calculated by calculating the index regarding the waveform of the unit section in each of the plurality of unit sections in which the blood flow signal is divided at each pulsation and averaging the indexes over a period of the plurality of unit sections. Accordingly, the index regarding the waveform in the plurality of unit sections can be smoothed and calculated as the blood flow index compared to a configuration in which index regarding the waveform in one unit section is calculated from the blood flow index. Further, whether there is the stenosis or atresia of the blood vessel can be determined with higher precision.

In the preferred aspect of the invention, the determination unit may determine whether there is the stenosis or atresia of the blood vessel according to a change amount of the blood flow index. In the above configuration, whether there is the stenosis or atresia of the blood vessel is determined according to the change amount in the blood flow index. For the blood flow index, there is a large individual difference depending on ages or health states. Accordingly, for example, in a configuration in which whether there is the stenosis or atresia of the blood vessel is determined in accordance with the comparison result of the blood flow index and the predetermined threshold, it is difficult to set an appropriate threshold and the problem that whether the stenosis or atresia of the blood vessel occurs may not be determined accurately is assumed. According to the above-described aspect of the invention, it is not necessary to set the threshold. Therefore, the problem that whether the stenosis or atresia of the blood vessel occurs may not be determined accurately is reduced.

In the preferred aspect of the invention, the measurement device may further include a report control unit that reports the stenosis or atresia of the blood vessel to a report device when the determination unit determines that the stenosis or atresia of the blood vessel occurs. In the above configuration, the stenosis or atresia of the blood vessel is reported when it is determined that the stenosis or atresia of the blood vessel occurs. Accordingly, the user can comprehend the stenosis or atresia of the blood vessel.

A measurement method according to another preferred aspect of the invention causes a computer to: calculate a blood flow index regarding a waveform of a blood flow signal indicating a temporal change in a blood flow rate of a subject; and determine whether there is stenosis or atresia of a blood vessel of the subject according to the blood flow index. In the above aspect of the invention, the same operations and advantages as those of the measurement device according to the aspect of the invention are realized.

### BRIEF DESCRIPTION OF THE DRAWINGS

The invention will be described with reference to the accompanying drawings, wherein like numbers reference like elements.

FIG. 1 is a side view illustrating a measurement device according to a first embodiment of the invention.

FIG. 2 is a diagram illustrating a configuration focusing on functions of the measurement device.

FIG. 3 is a diagram illustrating a blood flow signal indicating a temporal change in a blood flow rate of a subject.

FIG. 4 is a diagram illustrating a blood flow signal in a unit section.

FIG. 5 is a schematic diagram illustrating a blood vessel at the normal time.

FIG. 6 is a schematic diagram illustrating a blood vessel at the time of stenosis.

FIG. 7 is a flowchart illustrating a process of a control device.

### DESCRIPTION OF EXEMPLARY EMBODIMENTS

### First Embodiment

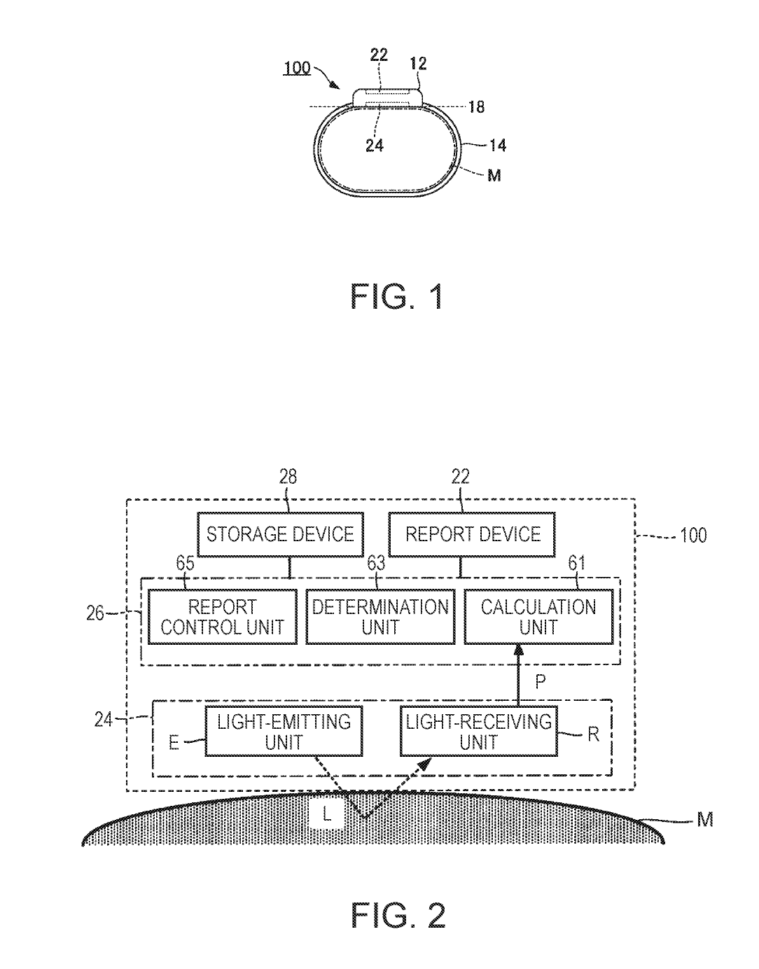

FIG. 1 is a side view illustrating a measurement device 100 according to a first embodiment of the invention. The measurement device 100 according to the first embodiment is a measurement device that evaluates a blood vessel state of a subject and is mounted on a part which is a measurement target in the body of the subject (hereinafter referred to as a “measurement part”) M. The measurement device 100 according to the first embodiment is a wristwatch type portable device including a casing unit 12 and a belt 14 and can be mounted on a wrist of the subject by winding the belt 14 around the wrist which is an example of the measurement part M.

The measurement device 100 according to the first embodiment evaluates whether there is stenosis or atresia of a blood vessel of the subject in the blood vessel. Stenosis or atresia of a blood vessel locally occurs due to blood clots or atheroma in the blood vessel. Atheroma A is amass occurring due to deposition of cholesterol or fat in blood to the membrane surface of a blood vessel F, as exemplified in FIG. 6. Here, stenosis or atresia of a blood vessel due to the occurrence of the atheroma A can cause cerebral infarction or cardiac infarction (hereinafter referred to as infarction). Specifically, as infarction caused by the atheroma A, there are thrombotic infarction, occlusive infarction, and hemodynamic infarction. The thrombotic infarction is caused when the atheroma A is burst and blood clots formed on the surface of the atheroma A occludes the blood vessel F. The occlusive infarction is caused when the blood clots formed on the surface of the atheroma A are extricated in blood and occlude the blood vessel F. The hemodynamic cerebral infarction is caused due to a reduction in a blood flow caused by a decrease in blood pressure or dehydration in a location in which the blood vessel F is stenosed by the atheroma A. As understood from the above description, it is particularly important to evaluate whether stenosis or atresia of a blood vessel occurs from the viewpoint of prevention of infarction of a subject.

FIG. 2 is a diagram illustrating a configuration focusing on functions of the measurement device 100. As exemplified in FIG. 2, the measurement device 100 according to the first embodiment includes a report device 22, a detection device 24, a control device 26, and a storage device 28. The control device 26 and the storage device 28 are installed inside the casing unit 12. The report device 22 is a display device (for example, a liquid crystal display panel) that is installed on the surface of the casing unit 12 (for example, the surface opposite to the measurement part M), as exemplified in FIG. 1 and displays various images under the control of the control device 26.

The detection device 24 in FIG. 2 is a sensor module that generates a detection signal P in accordance with a state of the measurement part M and is installed on, for example, the opposite surface 18 of the casing unit 12 (hereinafter referred to as a “detection surface”) to the measurement part M. The detection surface 18 is a flat surface or a curved surface. The detection device 24 according to the embodiment generates the detection signal P used to determine whether stenosis or atresia of a blood vessel occurs. As exemplified in FIG. 2, the detection device 24 includes a light-emitting unit E and a light-receiving unit R. The light-emitting unit E and the light-receiving unit R are installed on the detection surface 18 facing the measurement part M.

The light-emitting unit E in FIG. 2 emits light L to the measurement part M. For example, a light-emitting element that emits coherent light (that is, laser light) L with high coherence is used appropriately as the light-emitting unit E. As the light-emitting element that emits a laser, a vertical cavity surface emitting laser (VCSEL), a photonic crystal laser, a semiconductor laser, or the like can be applied. Here, a light-emitting diode (LED) can also be used as the light-emitting element.

The light L emitted from the light-emitting unit E is incident on the measurement part M, is repeatedly reflected and diffused inside the measurement part M, exits to the side of the detection surface 18, and arrive at the light-receiving unit R. That is, the light-emitting unit E and the light-receiving unit R function as a reflective optical sensor.

The light-receiving unit R generates a detection signal P according to a light reception level of the light arriving from the measurement part M. For example, a photoelectric conversion element such as a photodiode (PD) receiving the light L on the light reception surface facing the measurement part M is appropriately used as the light-receiving unit R. The detection device 24 includes, for example, a driving circuit that drives the light-emitting unit E when a driving current is supplied and an output circuit (for example, an amplification circuit and an A/D converter) that performs amplification and A/D conversion on an output signal of the light-receiving unit R. However, these circuits are not illustrated in FIG. 2.

The blood vessel F of the measurement part M expands and contracts repeatedly at a period equal to pulsation. A blood flow rate of the blood inside the blood vessel F differs between the time of expansion and the time of contraction. Therefore, the detection signal P generated according to the light reception level from the measurement part M by the light-receiving unit R is a pulse wave signal including a periodically varied component corresponding to a change in the blood flow rate of an artery of the measurement part M.

The control device 26 in FIG. 2 is an arithmetic processing device such as a central processing unit (CPU) or a field-programmable gate array (FPGA) and controls the entire measurement device 100. The storage device 28 is configured with, for example, a nonvolatile semiconductor memory and stores a program to be executed by the control device 26 and various kinds of data to be used by the control device 26. The control device 26 according to the first embodiment executes the program stored in the storage device 28 to realize a calculation unit 61, a determination unit 63, and a report control unit 65. A configuration in which functions of the control device 26 are distributed to a plurality of integrated circuits or a configuration in which some or all of the functions of the control device 26 are realized by dedicated electronic circuits can also be adopted. In FIG. 2, the control device 26 and the storage device 28 are illustrated as separate elements, but the control device 26 containing the storage device 28 can also be realized by, for example, an application specific integrated circuit (ASIC) or the like.

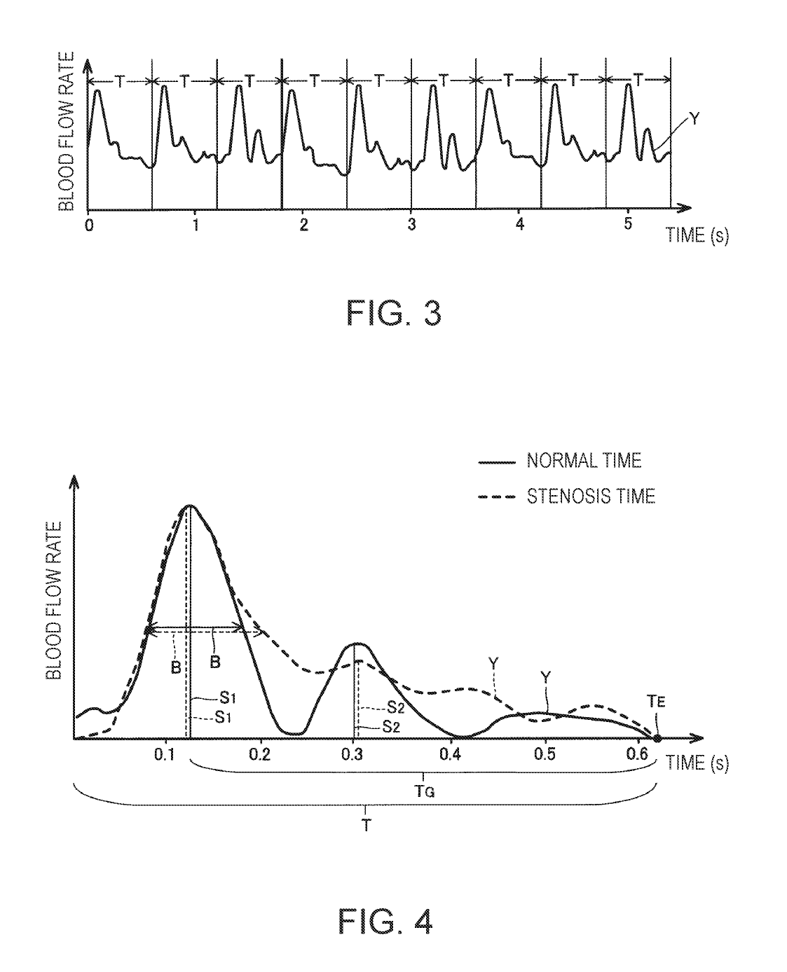

The calculation unit 61 calculates an index regarding a waveform of a blood flow signal Y indicating a temporal change in a blood flow rate of a subject (hereinafter referred to as a “blood flow index”). First, the calculation unit 61 generates the blood flow signal Y from the detection signal P generated by the detection device 24. Any known technology can be adopted to generate the blood flow signal Y. For example, the calculation unit 61 calculates a power spectrum from the detection signal P and calculates a blood flow rate from the calculated power spectrum. The calculation unit 61 generates a time series of the blood flow rate calculated at times different on a time axis as the blood flow signal Y. As exemplified in FIG. 3, the blood flow signal Y varies at a period with a time length (a period of about 0.5 seconds to 1 second) equivalent to one beat of pulsation. A section equivalent to each period of pulsation in the blood flow signal Y is referred to as a unit section T. That is, the unit section T is a section equivalent to one beat (one wavelength) of pulsation.

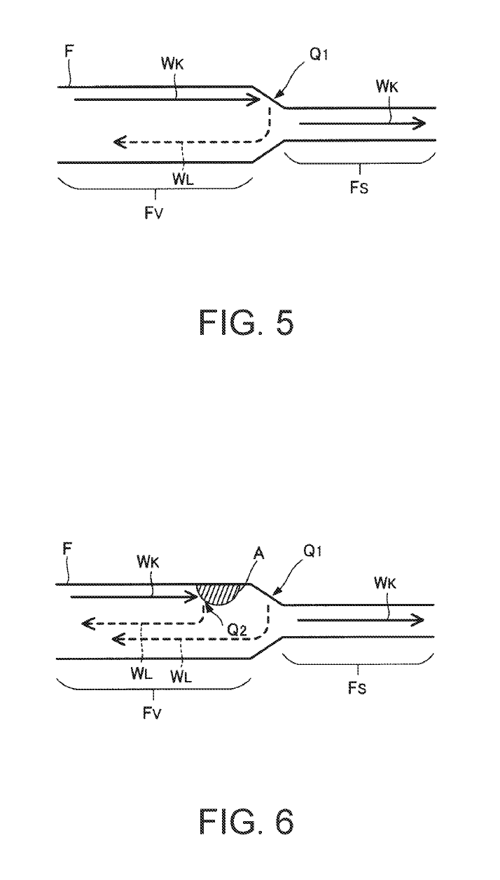

FIG. 4 is a diagram illustrating the waveform of the blood flow signal Y in the unit section T. In FIG. 4, a waveform (indicated by a solid line) of the blood flow signal Y measured at a normal time and a waveform (indicated by a dotted line) of the blood flow signal Y measured in a state in which a blood vessel is in a stenosis state (hereinafter referred to as a “stenosis time”) are illustrated together. When blood clots or the atheroma A occur, a cross-sectional area of the blood vessel F at the spot at which the blood clots or the atheroma A occur is contracted and is in the state in which the blood vessel is stenosed. That is, the blood flow signal Y at the stenosis time is the blood flow signal Y when the blood clots or the atheroma A is assumed to occur. Here, a pressure wave transmitted from a heart includes an orthodromic pressure wave (hereinafter referred to as an “ejection wave”) WK transmitted from the heart toward the peripheral vessel of the subject and an antidromic pressure wave (hereinafter referred to as a “reflected wave”) WL occurring when a part of the ejection wave WK is reflected at a reflection point Q inside the blood vessel F. as exemplified in FIG. 5.

A first peak of the blood flow signal Y in the unit section T mainly includes a component of the ejection wave WK and peaks after the second peak of the blood flow signal Y mainly include a component of the reflected wave WL. In the unit section T, the first peak has the largest strength and the second peak has the next largest strength of the first peak.

In FIG. 4, a hemline shape of the peak of the waveform of the blood flow signal Y in the unit section T is focused on. The hemline shape of the peak of the waveform in the first embodiment refers to a hemline shape of the first peak and specifically refers to a waveform at a period of a section TG from the center of the first peak to an end point TE of the unit section T. The waveform of a peak subsequent to the second peak is considered to have a hemline shape of the first peak for convenience.

The hemline shape of the peak of the waveform measured at the stenosis time differs from the hemline shape of the peak of the waveform at the normal time, as exemplified in FIG. 4. The hemline shape of the peak of the waveform at the stenosis time can be said to be continuous and gentle from the center of the first peak, compared to the hemline shape of the peak of the waveform at the normal time. In other words, the second peak of the waveform (that is, mainly the peak equivalent to the reflected wave WL) at the stenosis time is not clear compared to the second peak of the waveform at the normal time.

Hereinafter, the reason why the hemline shape of the peak of the waveform at the stenosis time is gentle compared to the hemline shape of the peak of the waveform at the normal time will be described. In the blood vessel F at the normal time (that is, when no stenosis or atresia of a blood vessel occurs), as exemplified in FIG. 5, the reflected wave WL is produced when the ejection wave WK is reflected at a reflection point Q1 which is a branch portion of an artery FV and a peripheral blood vessel FS. The reflected wave WL at the reflection point Q1 is mainly indicated in the section TG of the waveform at the normal time of FIG. 4. In contrast, in the blood vessel F at the stenosis time when the atheroma A occurs, as exemplified in FIG. 6, the reflected wave WL obtained by reflecting the ejection wave WK from the atheroma A is produced at a reflection point Q2 in addition to the reflected WL at the reflection point Q1. Since the reflected wave WL at the reflection point Q1 overlaps the reflected wave WL at the reflection point Q2, the hemline shape of the peak of the waveform at the stenosis time is continuous gentle from the center of the first peak, as exemplified in FIG. 4. Even at the stenosis time when the blood clots occur, the reflected wave WL is produced at the reflection point in the blood clots as at the stenosis time when the atheroma A occurs. Therefore, the hemline shape is continuously gentle from the center of the first peak. The reason why the hemline shape of the peak of the waveform at the stenosis time is gentle has been described above. The hemline shape of a peak of a waveform observed in a state in which a blood vessel is occluded (hereinafter referred to as the “atresia time”) also differs from the hemline shape of a peak of a waveform at the normal time, like the hemline shape of the peak of the waveform at the stenosis time in FIG. 4. The calculation unit 61 determines whether the stenosis or atresia of the blood vessel occurs based on a tendency of the gentle hemline shape of the peak of the waveform when stenosis or atresia of a blood vessel occurs due to blood clots or the atheroma A.

The calculation unit 61 calculates a blood flow index from the waveform of the generated blood flow signal Y. The calculation unit 61 according to the first embodiment calculates the blood flow index from the waveform of the plurality of unit sections T in which the blood flow signal Y is divided at each pulsation. Specifically, the calculation unit 61 calculates a blood flow index from a waveform obtained by averaging the waveforms of the plurality of unit sections T over a period of the plurality of unit sections T (hereinafter referred to as an “average waveform”). The calculation unit 61 specifies the average waveform in the blood flow signal Y over a predetermined period. The hemline shape of the peak of the waveform can vary due to a cause (for example, a cause such as temperature or a motion) other than stenosis or atresia of a blood vessel. Accordingly, the predetermined period in which the waveforms of the blood flow signal Y are averaged is set to be, for example, 3 or more hours in order exclude an influence of a cause other than stenosis or atresia of a blood vessel. In a process of dividing the blood flow signal Y into the unit sections T, any known technology can be adopted.

The hemline shape of the peak of the waveform in the unit section T becomes gentle when stenosis or atresia of a blood vessel occurs, as described above. Accordingly, the calculation unit 61 calculates the blood flow index according to the hemline shape of the peak of the average waveform. The blood flow index is calculated for each average waveform. Specifically, the calculation unit 61 calculates the blood flow index according to kurtosis Φ of the average waveform. That is, the kurtosis Φ is used as an index indicating the hemline shape. In the first embodiment, the calculation unit 61 calculates the kurtosis Φ as a blood flow index.

The kurtosis Φ can be an index indicating heaviness of the hemline shape (that is, the thickness of the hemline shape) at a peak of a waveform and sharpness of the peak of the waveform and can be, namely, an index indicating the degree of flatness of the peak. As a method of calculating the kurtosis Φ of the waveform in the unit section T, any known technology can be adopted. For example, the kurtosis Φ is expressed as in Equation (1) below. Here, n is the number of blood flow rates (data) indicating an average waveform, xi is a blood flow rate at a time i, xave is an average value of the blood flow rate in the unit section T, and s is a standard deviation. The calculation unit 61 causes the storage device 28 to store the calculated blood flow index.

$$Φ = \{ \frac{n ( n - 1 )}{( n - 1 ) ( n - 2 ) ( n - 3 )} \sum {( \frac{xi - {x}_{ave}}{s} )}^{4} \} - \frac{3 {( n - 2 )}^{2}}{( n - 2 ) ( n - 3 )} \qquad ( 1 )$$

As the kurtosis Φ is greater, the peak is sharper and the hemline shape is heavier. In contrast, as the kurtosis Φ is smaller, the peak is more rounded and the hemline shape is lighter (that is, the hemline shape of the peak of the waveform is gentler). As described above, the hemline shape of the peak of the waveform at the stenosis time or the atresia time is gentler than the hemline shape of the peak of the waveform at the normal time. Accordingly, the kurtosis Φ of the waveform at the stenosis time and the atresia time has a tendency to be smaller than the kurtosis Φ of the waveform at the normal time. For example, while the kurtosis Φ at the normal time is about 1.45, the kurtosis Φ at the stenosis time or the atresia time is about 1.04.

Based on the above tendency, the determination unit 63 determines whether stenosis or atresia of a blood vessel occurs according to the blood flow index (that is, the kurtosis Φ). The determination unit 63 according to the first embodiment determines whether stenosis or atresia of a blood vessel occurs according to a change amount of the blood flow index calculated by the calculation unit 61. Since it takes a time sufficient for a period of pulsation to grow the blood clots or the atheroma A, the determination unit 63 determines whether the stenosis or atresia of the blood vessel occurs according to a change amount from the blood flow index, for example, several hours before or one month before. As described above, since the kurtosis Φ decreases due to occurrence of the stenosis or atresia of the blood vessel, a decrease amount of kurtosis Φ is calculated as the change amount of the blood flow index. Specifically, the determination unit 63 determines whether the stenosis or atresia of the blood vessel occurs according to a comparison result obtained by comparing the change amount of blood flow index to a predetermined threshold. For example, when the change amount of blood flow index is greater than the predetermined threshold, the determination unit 63 determines that the stenosis or atresia of the blood vessel occurs. Conversely, when the change amount of blood flow index is less than the predetermined threshold, the determination unit 63 determines that no stenosis or atresia of the blood vessel occurs. The predetermined threshold is calculated experimentally or statistically.

The report control unit 65 in FIG. 2 causes the report device 22 to report various kinds of information. The report control unit 65 according to the first embodiment causes the report device 22 to report the occurrence of the stenosis or atresia of the blood vessel when the determination unit 63 determines that the stenosis or atresia of the blood vessel occurs. Specifically, the report control unit 65 causes the report device 22 to display an image to report the occurrence of the stenosis or atresia of the blood vessel. The report device 22 reports the occurrence of the stenosis or atresia of the blood vessel to the subject by displaying the image instructed by the report control unit 65.

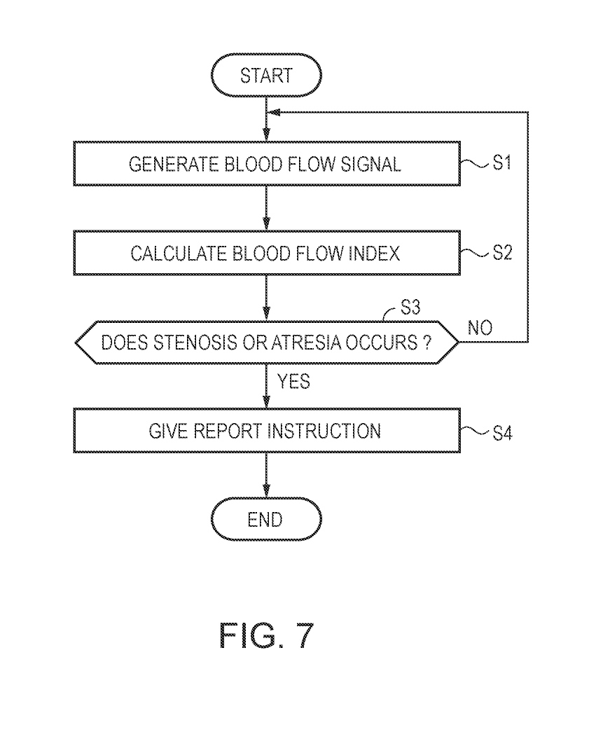

FIG. 7 is a flowchart illustrating a process of the control device 26. The process of FIG. 7 starts using an instruction to start measurement from the subject (activation of a program) as a trigger.

When the process of FIG. 7 starts, the calculation unit 61 generates the blood flow signal Y from the detection signal P generated by the detection device 24 (S1). The calculation unit 61 calculates the blood flow index from the generated blood flow signal Y (S2). Specifically, the calculation unit 61 calculates the kurtosis Φ of the average waveform of the blood flow signal Y as the blood flow index. Steps S1 and S2 are a process of calculating the blood flow index regarding the waveform of the blood flow signal. The determination unit 63 determines whether the stenosis or atresia of the blood vessel occurs according to the blood flow index (S3). When the change amount of the blood flow index is greater than the predetermined threshold (YES in S3), the determination unit 63 determines that the stenosis or atresia of the blood vessel occurs. The report control unit 65 instructs the report device 22 to report the occurrence of the stenosis or atresia (S4). Conversely, when the change amount of the blood flow index is less than the predetermined threshold (YES in S3), the process of steps S1 and S2 is repeated performed.

As understood from the above description, in the first embodiment, it is possible to determine whether the stenosis or atresia of the blood vessel of the subject occurs (that is, determine whether blood clots or the atheroma A occurs). In the first embodiment, in particular, it is possible to determine whether the stenosis or atresia of the blood vessel occurs with high precision based on the tendency that the kurtosis Φ of the waveform of the blood flow signal Y at the stenosis time or the atresia time is less than that at the normal time.

### Second Embodiment

A second embodiment of the invention will be described. The reference numerals used in the description of the first embodiment are used for the same elements as those of the first embodiment in operations and functions of each configuration to be exemplified below and the detailed description will be appropriately omitted.

The measurement device 100 according to the first embodiment determines whether stenosis or atresia of a blood vessel occurs from the blood flow index according to the kurtosis Φ of the waveform of the blood flow signal Y. The measurement device 100 according to the second embodiment determines whether stenosis or atresia of a blood vessel from a blood flow signal Y occurs according to a half width of the waveform of the blood flow signal Y. That is, in the second embodiment, the half width of the waveform is used as an index indicating a hemline shape. A half width B is an index indicating expansion of a peak of a waveform and refers to an interval (the width of a peak) between two time points at which a blood flow rate is a half of a maximum value (the center of the peak) of the peak, as exemplified in FIG. 4.

The calculation unit 61 according to the second embodiment calculates a blood flow index according to the half width B of the waveform of the blood flow signal Y. The calculation unit 61 calculates the half width B of the average waveform as a blood flow index. A process of specifying the average waveform is the same as the process of the first embodiment. The half width B according to the second embodiment is assumed to be the half width B of the first peak of the waveform in FIG. 4. Any known technology can be adopted to calculate the half width B. The calculation unit 61 causes the storage device 28 to store the calculated blood flow index as in the first embodiment.

Here, a hemline shape of the peak of a waveform (a waveform equivalent to the section TG) at the stenosis time in FIG. 4 is continuously gentle from the center of the first peak, as described above. Accordingly, the half width B of the waveform at the stenosis time has a tendency to be greater than the half width B of the waveform at the normal time, as exemplified in FIG. 4. Based on the above tendency, the determination unit 63 according to the second embodiment determines whether the stenosis or atresia of the blood vessel occurs according to the half width B. The determination unit 63 according to the second embodiment determines whether the stenosis or atresia of the blood vessel occurs according to a change amount of the blood flow index (the half width B) calculated by the calculation unit 61. As in the first embodiment, the determination unit 63 determines whether the stenosis or atresia of the blood vessel occurs according to a change amount from the blood flow index, for example, several hours before or one month before. As described above, since the half width B increases due to occurrence of the stenosis or atresia of the blood vessel, an increase amount of half width B is calculated as the change amount of the blood flow index. Specifically, the determination unit 63 determines whether the stenosis or atresia of the blood vessel occurs according to a comparison result obtained by comparing the change amount of blood flow index to a predetermined threshold. For example, when the change amount of blood flow index is greater than the predetermined threshold, the determination unit 63 determines that the stenosis or atresia of the blood vessel occurs. Conversely, when the change amount of blood flow index is less than the predetermined threshold, the determination unit 63 determines that no stenosis or atresia of the blood vessel occurs. The predetermined threshold is calculated experimentally or statistically.

The report control unit 65 according to the second embodiment causes the report device 22 to report the occurrence of the stenosis or atresia of the blood vessel when the determination unit 63 determines that the stenosis or atresia of the blood vessel occurs as in the first embodiment.

As understood from the above description, in the second embodiment, it is possible to determine whether the stenosis or atresia of the blood vessel F of the subject occurs as in the first embodiment. In the second embodiment, in particular, it is possible to determine whether the stenosis or atresia of the blood vessel occurs with high precision based on the tendency that the half width B of the waveform of the blood flow signal Y at the stenosis time or the atresia time is greater than that at the normal time.

### Third Embodiment

The measurement device 100 according to the first embodiment determines whether the stenosis or atresia of the blood vessel occurs based on the blood flow index according to the kurtosis Φ of the waveform of the blood flow signal Y. The measurement device 100 according to a third embodiment determines whether stenosis or atresia of a blood vessel occurs based on the blood flow index according to a ratio between to a first peak strength and a second peak strength in the unit section T. That is, in the third embodiment, the ratio between the first peak strength and the second peak strength is used as an index indicating the hemline shape.

The calculation unit 61 according to the third embodiment calculates a blood flow index according to the ratio between the first peak strength and the second peak strength in the unit section T. Specifically, the calculation unit 61 calculates a ratio S2/S1 of the second peak strength S2 to the first peak strength S1 (hereinafter referred to as a “peak strength ratio”) illustrated in FIG. 4 as a blood flow index. A process of specifying the average waveform is the same as the process of the first embodiment. The calculation unit 61 causes the storage device 28 to store the calculated blood flow index as in the first embodiment.

The hemline shape of the peak of the waveform (the waveform equivalent to the section TG in FIG. 4) at the time stenosis time and the atresia time differs from the hemline shape of the peak of the waveform at the normal time, as described above. The peak strength ratio S2/S1 at the stenosis time has a tendency to be less than the peak strength ratio S2/S1 at the normal time, as exemplified in FIG. 4. For example, while the peak strength ratio S2/S1 at the time normal time is about 0.4, the peak strength ratio S2/S1 at the time stenosis time is about 0.32.

Based on the above tendency, the determination unit according to the third embodiment determines whether stenosis or atresia of a blood vessel occurs according to the peak strength ratio S2/S1. Specifically, the determination unit 63 determines whether the stenosis or atresia of the blood vessel occurs according to a change amount of the blood flow index (the peak strength ratio S2/S1) calculated by the calculation unit 61. As in the first embodiment, the determination unit 63 determines whether the stenosis or atresia of the blood vessel occurs according to a change amount from the blood flow index, for example, several hours before or one month before. As described above, since the peak strength ratio S2/S1 decreases due to occurrence of the stenosis or atresia of the blood vessel, a decrease amount of peak strength ratio S2/S1 is calculated as the change amount of the blood flow index. Specifically, the determination unit 63 determines whether the stenosis or atresia of the blood vessel occurs according to a comparison result obtained by comparing the change amount of blood flow index to a predetermined threshold. For example, when the change amount of blood flow index is greater than the predetermined threshold, the determination unit 63 determines that the stenosis or atresia of the blood vessel occurs. Conversely, when the change amount of blood flow index is less than the predetermined threshold, the determination unit 63 determines that no stenosis or atresia of the blood vessel occurs. The predetermined threshold is calculated experimentally or statistically.

The report control unit 65 according to the third embodiment causes the report device 22 to report the occurrence of the stenosis or atresia of the blood vessel when the determination unit 63 determines that the stenosis or atresia of the blood vessel occurs as in the first embodiment.

As understood from the above description, in the third embodiment, it is possible to determine whether the stenosis or atresia of the blood vessel F of the subject occurs as in the first embodiment. In the third embodiment, in particular, it is possible to determine whether the stenosis or atresia of the blood vessel occurs with high precision based on the tendency that the peak strength ratio S2/S1 at the stenosis time or the atresia time is less than that at the normal time.

### Modification Examples

The above-exemplified embodiments can be modified variously. Specific modification aspects will be exemplified below. Two or more aspects arbitrarily selected from the following examples can also be appropriately combined.

(1) In the above-described embodiments, the various indexes according to the hemline shape have been calculated as a blood flow index since the hemline shape of the peak of the waveform at the stenosis time or the atresia time differs from that at the normal time. The index of the hemline shape available as the blood flow index is not limited to the examples (the kurtosis Φ, the half width B, and the peak strength ratio S2/S1) according to the above-described embodiments. For example, a Q value of the peak can also be calculated as the blood flow index. That is, the calculation unit 61 can be expressed comprehensively as an element calculating a blood flow index according to the hemline shape of the peak of the waveform of the blood flow signal Y.

(2) In the above-described embodiments, the various indexes (the kurtosis Φ, the half width B, and the peak strength ratio S2/S1) according to the hemline shape have been selected as the blood flow indexes, but a relation between the index of the hemline shape and the blood flow index is not limited to the above examples (the indexes of the hemline shape=the blood flow indexes). For example, it is also possible to calculate the blood flow index through predetermined calculation using the index of the hemline shape. For example, a reciprocal of the index of the hemline shape or a value obtained by adding or subtracting the index to or from a predetermined numerical value can also be calculated as the blood flow index.

(3) In the above-described embodiments, the blood flow index has been calculated from the waveform of the plurality of unit sections T in which the blood flow signal Y is divided at each pulsation. However, the blood flow index can also be calculated from the waveform of one unit section T. That is, the calculation unit 61 can include both of an element calculating a blood flow index from the average waveform and an element calculating a blood flow index according to the waveform in one unit section T and can be expressed comprehensively as an element calculating a blood flow index according the waveform in the unit section T.

(4) In the above-described embodiments, the blood flow index has been calculated according to the waveform in the unit section T, but the section of the waveform used to calculate the blood flow index is not limited to the above example. The blood flow index can also be calculated according to a waveform in a section longer than unit section T or a section shorter than the unit section T. Here, in the configuration in which the blood flow index is calculated according to the waveform in the unit section T, whether the stenosis or atresia of the blood vessel occurs can be determined with high precision based on the tendency that a change in the waveform according to whether there is the stenosis or atresia of the blood vessel is more remarkable at one beat of pulsation.

(5) In the above-described embodiments, the configuration in which the blood flow index is calculated from the average waveform obtained by averaging the waveforms of the plurality of unit sections T in which the blood flow signal Y is divided at each pulsation over the period of the plurality of unit sections T (hereinafter referred to as “Configuration 1”) has been adopted, but the method of calculating the blood flow index is not limited to the above example. For example, a configuration in which the blood flow index is calculated by calculating an index regarding the waveform in the unit section T for each of the plurality of unit sections T and averaging the indexes over a period of the plurality of unit sections T (hereinafter referred to as “Configuration 2”) can also be used. In Configuration 1, a blood flow index can be calculated in accordance with a smoothed waveform. In Configuration 2, an index regarding a waveform in the plurality of unit sections T can be smoothed to be calculated as a blood flow index. In both Configurations 1 and 2, whether stenosis or atresia of a blood vessel occurs can be determined with higher precision compared to a configuration in which a blood flow index is calculated from a waveform in one unit section T.

(6) In the above-described embodiments, the waveform over the section TG from the center of the first peak to the endpoint TE of the unit section T has been used as the hemline shape, but the hemline shape is not limited to the waveform over the section TG. Any hemline shape can be used as long as the hemline shape is a waveform in a section included in the section TG. That is, the positions of a start point and an end point of a section equivalent to the hemline shape do not matter.

(7) In the above-described embodiments, whether the stenosis or atresia of the blood vessel occurs has been determined according to the change amount of blood flow index. However, whether stenosis or atresia of a blood vessel occurs can also be determined according to a comparison result of the blood flow index and the predetermined threshold. Here, for the blood flow index, there is a large individual difference depending on ages or health states. Accordingly, in a configuration in which whether there is stenosis or atresia of a blood vessel is determined in accordance with the comparison result of the blood flow index and the predetermined threshold, it is difficult to set an appropriate threshold and the problem that whether stenosis or atresia of a blood vessel occurs may not be determined accurately is assumed. According to the above-described embodiments in which whether stenosis or atresia of a blood vessel occurs is determined according to a change amount of blood flow index, it is not necessary to set the threshold. Therefore, the problem that whether stenosis or atresia of a blood vessel occurs may not be determined accurately is reduced.

(8) In the third embodiment, the ratio of the second peak strength to the first peak strength has been calculated as the blood flow index, but a ratio of the first peak strength to the second peak strength can also be calculated as a blood flow index.

(9) In the above-described embodiments, the report control unit 65 causing the report device 22 to report the occurrence of the stenosis or atresia of the blood vessel when the determination unit 63 determines that the stenosis or atresia of the blood vessel occurs is included, but the report control unit 65 is not a requisite in the invention. According to the above-described embodiments in which the occurrence of the stenosis or atresia of the blood vessel is reported, the user can comprehend the occurrence of the stenosis or atresia of the blood vessel. Further, diseases such as infarction can be usefully prevented.

(10) In the above-described embodiments, the display device displaying an image to report the occurrence of stenosis or atresia of a blood vessel has been configured as the report device 22. However, a sound emitting device (for example, a speaker) that emits a sound to report the occurrence of the stenosis or atresia of the blood vessel can also be used as a report device.

(11) In the above-described embodiments, the report device 22 of the measurement device 100 reports the occurrence of the stenosis or atresia of the blood vessel to the subject. However, the occurrence of the stenosis or atresia of the blood vessel can also be reported to a report device separated from the measurement device 100. For example, the occurrence of the stenosis or atresia of the blood vessel can be reported to a report device of a terminal device (for example, a mobile phone or a smartphone) capable of communicating with the measurement device 100. Here, when a subject is the aged, there is a possibility that a disease such as infarction caused due to stenosis or atresia of a blood vessel is found late. In a configuration in which the occurrence of the stenosis or atresia of the blood vessel is reported to the report device separated from the measurement device 100, a disease can be earlier detected by reporting the occurrence of the stenosis or atresia of the blood vessel to a report device of a terminal device of a family member of a subject, a neighborhood of the subject, or a medical staff of a hospital which the subject goes to. The above configuration is particularly effective when the subject is the aged.

(12) In the above-described embodiments, the single measurement device 100 has performed the calculation of the blood flow index, the determination of whether the stenosis or atresia of the blood vessel occurs, and the instruction to report the occurrence of the stenosis or atresia of the blood vessel. However, the functions of the measurement device 100 can also be realized by a plurality of devices, as exemplified in the above-described embodiments. For example, the calculation of the blood flow index, the determination of whether the stenosis or atresia of the blood vessel occurs, and the report of the occurrence of the stenosis or atresia can also be realized using a terminal device capable of communicating the detection device 24 as the measurement device 100. Specifically, the detection signal P generated by the detection device 24 is transmitted to the terminal device. The terminal device determines whether stenosis or atresia of a blood vessel occurs from the detection signal P received from the detection device 24 and causes the report device of the terminal device to report the occurrence of the stenosis or atresia of the blood vessel. As understood from the above example, the detection device 24 and the control device 26 may be configured to be separated from each other.

One or a plurality of the calculation unit 61, the determination unit 63, and the report control unit 65 may be configured to be included in the terminal device (for example, a configuration in which an application is executed on the terminal device). As understood from the above description, the measurement device 100 can also be realized a plurality of devices separated from each other.

(13) In the above-described embodiments, the measurement device 100 configured to include the belt 14 and the casing unit 12 has been exemplified, but any specific type of the measurement device 100 can be realized. For example, any type of measurement device 100, such as a patch type of device which can be attached to the body of a subject, an earring type of device which can be mounted on an auricle of the subject, a finger-mounted type (for example, a nail type) of device which can be mounted on a fingertip of the subject, or a head-mounted type of device which can be mounted on the head of the subject, can be adopted. Here, for example, since there is a possibility of a problem occurring in a daily life in a state in which the measurement device 100 such as a finger-mounted type of device is mounted, the above-described type of measurement device 100 which can be mounted on the wrist of the subject by the belt 14 is particularly suitable from the viewpoint of generating the detection signal P normally without a problem in a daily life. A type of measurement device 100 which can be mounted on any of various electronic apparatuses such as a wristwatch can also be realized.

(14) The invention can also be specified as a method (measurement method) of operating the measurement device 100. Specifically, the measurement method according to a preferred aspect of the invention causes a computer to calculate a blood flow index regarding the waveform of the blood flow signal Y indicating a temporal change in a blood flow rate of the subject and to determine whether stenosis or atresia of the blood vessel F of the subject occurs according to the blood flow index. The entire disclosure of Japanese Patent Application No. 2016-207615 is hereby incorporated herein by reference.

What is claimed is:

1. A measurement device comprising:
a calculation unit that calculates a blood flow index regarding a waveform of a blood flow signal indicating a temporal change in a blood flow rate of a subject; and
a determination unit that determines whether there is stenosis or atresia of a blood vessel of the subject according to the blood flow index.

2. The measurement device according to claim 1,
wherein the calculation unit calculates the blood flow index according to a hemline shape of a peak of the waveform.

3. The measurement device according to claim 2,
wherein the calculation unit calculates the blood flow index according to kurtosis of the waveform.

4. The measurement device according to claim 2,
wherein the calculation unit calculates the blood flow index according to a half width of the waveform.

5. The measurement device according to claim 1,
wherein the calculation unit calculates the blood flow index according to a waveform in a unit section equivalent to one beat of pulsation in the blood flow signal.

6. The measurement device according to claim 2,
wherein the calculation unit calculates the blood flow index according to a ratio between a first peak strength and a second peak strength in a unit section equivalent to one beat of pulsation in the blood flow signal.

7. The measurement device according to claim 5,
wherein the calculation unit calculates the blood flow index from an average waveform obtained by averaging waveforms of a plurality of unit sections in which the blood flow signal is divided at each pulsation over a period of the plurality of unit sections.

8. The measurement device according to claim 5,
wherein the calculation unit calculates the blood flow index by calculating an index regarding a waveform of the unit section in each of the plurality of unit sections in which the blood flow signal is divided at each pulsation and averaging the indexes over a period of the plurality of unit sections.

9. The measurement device according to claim 1,
wherein the determination unit determines whether there is the stenosis or atresia of the blood vessel according to a change amount of the blood flow index.

10. The measurement device according to claim 1, further comprising:
a report control unit that reports the stenosis or atresia of the blood vessel to a report device when the determination unit determines that the stenosis or atresia of the blood vessel occurs.

11. A measurement method of causing a computer to:
calculate a blood flow index regarding a waveform of a blood flow signal indicating a temporal change in a blood flow rate of a subject; and
determine whether there is stenosis or atresia of a blood vessel of the subject according to the blood flow index.
